(19) United States
(12) Patent Application Publication
(10) Pub. No.: US 2004/0176453 A1
(43) Pub. Date: Sep. 9, 2004

(54) Method of treating time-dependent changes in human subjects

(76) Inventor: Arthur Vanmoor, Boca Raton, FL (US)

Correspondence Address: OTTO S. KAUDER, 5382C Venetia Ct., Boynton Beach, FL, 33437, US

(21) Appl. No.: 10/378,525

(22) Filed: Mar. 3, 2003

(51) Int. Cl. A61K 31/198

(52) U.S. Cl. 514/561; 514/563

(57) ABSTRACT

There is disclosed a method of treating manifestations of time-dependent change such as wrinkles, change of hair color and diminution of physical capabilities in a person in need of such treatment, which comprises the oral administration to such person of an effective amount of at least one water-soluble aminoacid represented by formula (I)

X—CₙH₂ₙ—CR(NH₂)COOH (I)

wherein X represents a hydrogen atom (H) or a carbamoyl group (CONH₂), R is hydrogen or a methyl (CH₃) group, and n is a whole number from 1 to 4.

### BACKGROUND OF THE INVENTION

1. Field of the Invention

This invention relates to a method of treating a person manifesting time-dependent changes for example skin wrinkles, changes in hair color, loss of hair, or loss of memory with an agent that enhances the effectiveness of the human immune system to mitigate and where possible eliminate the effects of aging.

2. Description of Related Art

Manifestations of time dependent changes are found everywhere, in living nature, in inanimate nature and man-made materials, in people. Flowers fade, foliage changes color, rocks weather and erode. Articles made of leather, paper, plastics and rubber embrittle slowly with time. The changes with time that people observe in themselves and other people are too well known to require detailing.

Some changes with time, as in fine wines, cheeses, and young people growing into mature adults, are viewed favorably. Most manifestations of change with time, however, are believed to be unfortunate and humankind has long endeavored to slow down, mitigate, redirect, and if possible arrest and reverse such manifestations, and to gain the understanding to enable advances in this endeavor to be realized.

The human immune system functions to maintain human individuality by fighting off foreign entities. The MERCK MANUAL, 16^{th} edition, published 1992, at pages 279 to 303, which portion is here incorporated by reference, contains a detailed description of the parts of the immune system and of immunodeficiency diseases and hypersensitivity disorders to which it is subject. A table at pages 284-5 titled “Cytokines” lists the major effects of such cytokines or immunoeffective polypeptides as interleukin types, interferon types, alpha- and beta-tumor necrosis factor, three types of colony-stimulating factor, and alpha- and beta-transforming growth factor. A table at page 303 lists disorders with increased susceptibility to unusual infections. Nothing in this publication relates to such time-dependent effects as aging the skin or remedies therefor.

Yu and Van Scott U.S. Pat. No. 5,091,771 disclosed a method of retarding appearance of wrinkles on human skin by treating the skin with a composition comprising an alpha-hydroxy carboxylic acid such as hydroxyacetic acid and a pH-buffering amino acid minimizing the aggressiveness of the alpha-hydroxy carboxylic acid on the skin. Nothing is disclosed relating to oral administration of the disclosed composition. Moreover, hydroxyacetic acid and certain other disclosed alpha-hydroxy carboxylic acids are too toxic to be considered for oral administration.

Hildebrand U.S. Pat. No. 5,296,500 disclosed a method for regulating wrinkles and/or atrophy in mammalian skin comprising treating the skin with a safe and effective amount of N-acetyl-L-cysteine and/or a derivative thereof. All compositions disclosed for this purpose are topical compositions.

Published PCT application PCT/US01/00714 discloses treatment of an ailment, which can be aging of the human skin, with an aliphatic sulfur compound.

### SUMMARY OF THE INVENTION

In accordance with this invention, there is provided a method of treating a manifestation of time-dependent change in a person in need of such treatment, which comprises the oral administration to such person of an effective amount of at least one water-soluble aminoacid represented by formula (I) shown below.

X—CₙH₂ₙ—CR(NH₂)COOH (I)

In formula (I), X represents a hydrogen atom (H) or a carbamoyl group (CONH₂), R is hydrogen or a methyl (CH₃) group, and n is a whole number from 1 to 4. When n is greater than 1, the alkylene group represented by CₙH₂ₙ can be straight chain or branched.

Without intending to be bound by any theory, it is believed that the present invention is can be beneficial in augmenting the person's innate ability to eliminate toxins from the body and to resist the initiation of the process that leads to development of wrinkles on human skin, and other manifestations of time-dependent changes, including changes in the color or quantity of hair, or diminution of physical capabilities such as walking, running, bicycling, climbing, weight lifting, and gymnasitic exercises, as well as to slow down, arrest, and even reverse that process. As a result, the incidence of facial wrinkles, dry skin an other aging related skin disorders, gray hair, progressive hair loss, memory loss, decrease in physical skills, and psychological discomfort associated with such changes is diminished, and the quality of life is improved.

In lessening manifestations of such time dependent changes as wrinkles and other age-related skin disorders mega-nutrient doses of 2 to 20 grams of at least one water-soluble aminoacid represented by formula (I) can be administered from one to five times daily, for a total of 2 to 100 grams per day, until monitoring shows sufficient improvement in the user's condition to permit reduction in dose level and ultimately cessation of the treatment. Daily doses of at least one water-soluble aminoacid represented by formula (I) in the range from 2 to 40 grams per day are preferred. Such doses can be administered in any convenient manner, as by oral administration in any of the usual dosage forms, such as tablets, capsules, solutions, and dispersions in liquid foods such as soups and fruit juices.

In formula (I), when n=1 the alkylene group represented by CₙH₂ₙ is a methylene group. When n=2, the alkylene group can be ethylene —CH₂CH₂— or ethylidene —CH(CH₃)—. When n=3 and n=4, the alkylene group represented by CnH₂ₙ, can have each of the known isomeric configurations of such straight chain and branched alkylene groups.

The following table contains preferred water-soluble amino acids represented by formula (I) according to the invention.

| Index | Name | X | R | n |
|---|---|---|---|---|
| 1 | 2-aminobutanoic acid | H | H | 2 |
| 2 | 2-amino-2-methylpropanoic acid | H | CH₃ | 1 |
| 3 | 2-amino-3-carbamoylpropanoic acid | CONH₂ | H | 1 |
| 4 | 2-amino-4-carbamoylbutanoic acid | CONH₂ | H | 2 |
| 5 | 2-amino-3-methylpentanoic acid | H | H | 4 |
| 6 | 2-amino-4-methylpentanoic acid | H | H | 4 |
| 7 | 2-amino-3-methylbutanoic acid | H | H | 3 |

### EXAMPLE 1

A bearded male volunteer aged 45 years whose beard had become close to 10% gray started a program of taking 40 grams of a combination of compounds represented by formula I in three doses of approximately 10, 10, and 20 grams respectively with the morning, midday, and evening meals. After six months of this program his beard was entirely black.

### EXAMPLE 2

A 41 year old male took 100 grams daily of composition containing compounds of formula (I) for a period of eight months. After this period he has been told by persons who saw him only before and after the treatment period that his appearance suggested that a face lift had been obtained. Also, comparison of photographs before and after the treatment period showed a younger appearance after the treatment with disappearance of wrinkles and softer texture of the skin. His overall energy level has increased to the level of ten years earlier. All this was achieved without changes in diet or physical activity.

### EXAMPLE 3

A fifty year old man bicycled the same five mile course daily for many years. From age 47 to age 50 his time to complete the course increased from 12 minutes to 20 minutes. At age 50 he started taking 50 grams each day of compound represented by formula (I). After 5 months of this treatment his time to bicycle the 5 mile course had diminished to 15 minutes.

What is claimed is:

1. A method of treating a manifestation of time dependent change in a person in need of such treatment, which comprises the oral administration to such person of an effective amount of at least one water-soluble amino acids represented by formula (I)
X—CₙH₂ₙ—CR(NH₂)COOH (1)
wherein X represents a hydrogen atom (H) or a carbamoyl group (CONH₂), R is hydrogen or a methyl (CH₃) group, and n is a whole number from 1 to 4.

2. The method of claim 1, wherein the effective amount of the aminoacid is administered in one to five daily doses, each dose being in the range of 2 to 20 gram.

3. The method of claim 1, wherein the total of said effective amount of the aminoacid administered daily is in the range of 2 to 100 grams.

4. The method of claim 3 wherein the total of said effective amount of the aminoacid administered daily is in the range of 20 to 50 grams.

5. The method of claim 1, wherein said person experiences lessening of the manifestation of time-dependent change.

6. The method of claim 1 wherein the aminoacid is 2-amino-4-carbamoylbutanoic acid.

7. The method of claim 1 wherein the aminoacid is 2-aminobutanoic acid

8. The method of claim 1 wherein the aminoacid is 2-amino-2-methylpropanoic acid

9 The method of claim 1 wherein the aminoacid is 2-amino-3-carbamoylpropanoic acid

10. The method of claim 1 wherein the aminoacid is 2-amino-3-methylpentanoic acid

11. The method of claim 1 wherein the aminoacid is 2-amino-4-methylpentanoic acid

12. The method of claim 1 wherein the aminoacid is 2-amino-3-methylbutanoic acid

13. The method of claim 1 wherein a plurality of water-soluble aminoacids represented by formula (I) is administered.

14. The method of claim 1, wherein the manifestation of time dependent change is wrinkling of the skin.

15. The method of claim 1, wherein the manifestation of time dependent change is change in hair color.

16. The method of claim 1, wherein the manifestation of time dependent change is diminution of physical capability.
